(19) United States
(12) Patent Application Publication
(10) Pub. No.: US 2005/0182488 A1
(43) Pub. Date: Aug. 18, 2005

(54) Implant and method for altering the refractive properties of the eye

(76) Inventor: Gholam A. Peyman, New Orleans, LA (US)

Correspondence Address: Bell, Boyd & Lloyd LLC, P.O. Box 1135, Chicago, IL, 60690-1135, US

(21) Appl. No.: 11/108,048

(22) Filed: Apr. 15, 2005

Related U.S. Application Data
(63) Continuation-in-part of application No. 10/784,169, filed on Feb. 24, 2004.
Application No. 10/784,169 is a continuation-in-part of application No. 10/406,558, filed on Apr. 4, 2003.
Application No. 10/784,169 is a continuation-in-part of application No. 10/356,730, filed on Feb. 3, 2003.
Application No. 10/356,730 is a continuation-in-part of application No. 09/843,141, filed on Apr. 27, 2001, now Pat. No. 6,551,307.
(60) Provisional application No. 60/449,617, filed on Feb. 26, 2003.

(51) Int. Cl. A61F 2/14; A61F 9/013

(52) U.S. Cl. 623/5.12; 623/5.11; 606/107; 606/166

(57) ABSTRACT

The present invention relates an implant and method for changing the refractive properties of an eye. The implant includes a body adapted to be implanted between layers of the cornea offset from the main optical axis, thereby changing the refractive properties of the eye. The body includes a first portion having a first refractive power and configured to change the refractive properties of a first area of the cornea by changing the corneal curvature thereof, and a second portion having a second refractive power, the second refractive power configured to change the refractive properties of a second area of the cornea and compensate for error at the second area caused by the first portion.

### CROSS-REFERENCE TO RELATED APPLICATION

This application is a continuation-in-part of U.S. patent application Ser. No. 10/784,169, filed Feb. 24, 2004, which is a continuation-in-part of U.S. patent application Ser. No. 10/406,558, filed Apr. 4, 2003, which claims the benefit of U.S. Provisional Application Ser. No. 60/449,617, filed Feb. 26, 2003 and is a continuation-in-part of U.S. Application Ser. No. 10/356,730, filed Feb. 3, 2003, which is a continuation-in-part of U.S. patent application Ser. No. 09/843,141, filed Apr. 27, 2001, now U.S. Pat. No. 6,551,307. The entire contents of each of which are herein incorporated by reference.

This application is related to U.S. application Ser. No. ______, entitled BIFOCAL IMPLANT AND METHOD FOR ALTERING THE REFRACTIVE PROPERTIES OF THE EYE, filed Apr. 15, 2005, the entire contents of which are herein incorporated by reference.

### BACKGROUND

A conventional method for correcting the refractive error in a cornea is keratophakia, i.e., implantation of a lens inside the cornea. Keratophakia uses an implant which is placed into the cornea approximately equidistant from the exterior surface of the cornea and the interior surface. The procedure is usually done by first preparing a lens from corneal donor tissue or synthetic material using a cryo-lathe. The lens is implanted by removing a portion of the cornea with a device called a microkeratome, and the tissue is sutured back into place over the lens. However, there can be problems when microkeratomies are used for cutting the cornea. First, irregular keratectomies or perforations of the eye can result. Second, the recovery of vision can be rather prolonged.

Additional surgical techniques exist that use ultraviolet light and short wavelength lasers to modify the shape of the cornea. For example, excimer lasers, such as those described in U.S. Pat. No. 4,840,175 to Peyman, which emit pulsed ultraviolet radiation, can be used to decompose or photoablate tissue in the live cornea so as to reshape the cornea.

Specifically, the Peyman patent discloses the laser surgical technique known as laser in situ keratomycosis (LASIK). In this technique, a portion of the front of the live cornea can be cut away in the form of a flap having a thickness of about 160 microns. This cut portion is removed from the live cornea to expose an inner surface of the cornea. A laser beam is then directed onto the exposed inner surface to ablate a desired amount of the inner surface up to 150-180 microns deep. The cut portion is reattached over the ablated portion of the cornea and assumes a shape conforming to that of the ablated portion

However, because only certain amount of cornea can be ablated without the remaining cornea becoming unstable or experiencing outbulging (ectasia), this technique is not especially effective in correcting very high myopia. That is, a typical cornea is on average about 500 microns thick. The laser ablation technique requires that at least about 250 microns of the corneal stroma remain after the ablation is completed so that instability and outbulging do not occur. Also, these conventional implants, while correcting a refractive error of the patient, also distort the normal vision of the patient.

Additional methods for correcting the refractive error in the eye include inserting an implant in-between layers of the cornea. Generally, this is achieved using several different methods. The first method involves inserting a ring between layers of the cornea, as described in U.S. Pat. No. 5,405,384 to Silvestrini. Typically, a dissector is inserted in the cornea and forms a channel therein. Once it is removed, a ring is then inserted into the channel to alter the curvature of the cornea. In the second method, a flap can be created similarly to the LASIK procedure and a lens can be inserted under the flap, as described in U.S. Pat. No. 6,102,946 to Nigam. The third method involves forming a pocket using an instrument, and inserting an implant into the pocket, as described in U.S. Pat. No. 4,655,774 to Choyce.

However, with the above described techniques, a knife or other mechanical instrument is generally used to form the channel, flap or pocket. Use of these instruments may result in damage or imprecision in the cut or formation of the desired area in which the implant is placed. Additionally, these conventional techniques do not include determination and testing of an appropriate implant for correcting a refractive error of a particular patient.

Therefore, there exists a need for an inlay and improved method of correcting refractive error in the cornea of an eye.

### SUMMARY

In one embodiment, a method of changing the refractive properties of an eye is provided. The method includes the step of separating the cornea to form a first layer and a second layer, the first layer facing in a posterior direction of the eye and the second layer facing in an anterior direction of the eye. The first and second layers preferably remain attached at an area at the main optical axis. An implant is then inserted between the first and second layers. The implant includes a first portion having a first index of refraction and adapted to change the refractive properties of a first portion of the eye, a second portion having a second index of refraction and adapted to change the refractive properties of a second portion of the eye, and a third portion having a substantially arcuate edge adapted to be positioned adjacent the area attached at the main optical axis.

In another embodiment, an implant for changing the refractive properties of an eye is provided. The implant includes a body adapted to be implanted between layers of the cornea offset from the main optical axis, thereby changing the refractive properties of the eye. The body has a first portion with a first refractive power and configured to change the refractive properties of a first area of the cornea by changing the corneal curvature thereof and a second portion with a second refractive power. The second refractive power is configured to change the refractive properties of a second area of the cornea and compensate for error at the second area caused by the first portion.

In another embodiment, an implant for changing the refractive properties of an eye is provided. The implant includes a first semi circular body portion adapted to be implanted between layers of the cornea offset from the main optical axis, thereby changing the refractive properties of the eye. The first semi circular body portion includes a first portion having a first refractive power and configured to change the refractive properties of a first area of the cornea by changing the corneal curvature thereof and a second portion having a second refractive power, said second refractive power configured to change the refractive properties of a second area of the cornea and compensate for error at the second area caused by the first portion. The implant further includes a second semi circular body portion adapted to be implanted between layers of the cornea offset from the main optical axis, thereby changing the refractive properties of the eye. The second semi circular body portion includes a third portion having a third refractive power configured to change the refractive properties of a third area of the cornea by changing the corneal curvature thereof and a fourth portion having a fourth refractive power, the fourth refractive power configured to change the refractive properties of a fourth area of the cornea and compensate for error at the second area caused by the third portion.

In another embodiment, a method of changing the refractive properties of the an eye is provided. The method includes the step of separating the cornea to form a first layer and a second layer, the first layer facing in a posterior direction of the eye and the second layer facing in an anterior direction of the eye, the first and second layers remaining attached at an area at the main optical axis. A first semi-circular implant is inserted between the first and second layers, the first implant including a first portion having a first index of refraction and adapted to change the refractive properties of a first portion of the eye, and a second portion having a second index of refraction and adapted to change the refractive properties of a second portion of the eye. A second semi-circular implant is inserted between said first and second layers, the second implant including a third portion having a third index of refraction and adapted to change the refractive properties of a third portion of the eye, a fourth portion having a fourth index of refraction and adapted to change the refractive properties of a fourth portion of the eye.

Other objects, advantages, and novel salient features of the present invention will become apparent to those skilled in the art from the following detailed description, which, taken in conjunction with the annexed drawings, discloses preferred embodiments of the invention.

### BRIEF DESCRIPTION OF THE DRAWINGS

Referring to the drawings which form a part of this disclosure:

FIG. 1 is a side view in section taken along the center of a myopic eye, showing the cornea, pupil and lens;

FIG. 2 is a side view in section of the eye of FIG. 1 with a flap formed in the surface of the cornea;

FIG. 3 is a side view in section of the eye of FIG. 2 with the periphery of the flap lifted up;

FIG. 4a is a top view of an implant according to an embodiment of the present invention;

FIG. 4b is a top view of an implant according to another embodiment of the present invention;

FIG. 5 is a cross-sectional side view of the implant of FIG. 4 taken along lines 5-5;

FIG. 6 is a side view in section of the eye of FIG. 3 with the implant of FIG. 4a inserted under the flap;

FIG. 7 is a top view of an eye having a corneal flap formed therein with the implant of FIG. 4b positioned under the flap;

FIG. 8 is a side view in section of an implant according to another embodiment of the present invention;

FIG. 9 is a side view in section of the eye of FIG. 3 with the implant of FIG. 7 inserted under the flap;

FIG. 10 is a side view in section of the eye of FIG. 6 with a second flap formed in the surface of the cornea;

FIG. 11 is a side view in section of the eye and implant of FIG. 9 with an additional implant inserted under the second flap;

FIG. 12 is a side view in section of the eye and implants of FIG. 10 with the second flap repositioned over its respective implant;

FIG. 13 is a side view in section of the eye and implant of FIG. 9 with a laser ablating a portion of the cornea; and

FIG. 14 is a side view in section of the eye and implant of FIG. 12 with the second flap repositioned over the ablated portion of the cornea.

### DETAILED DESCRIPTION

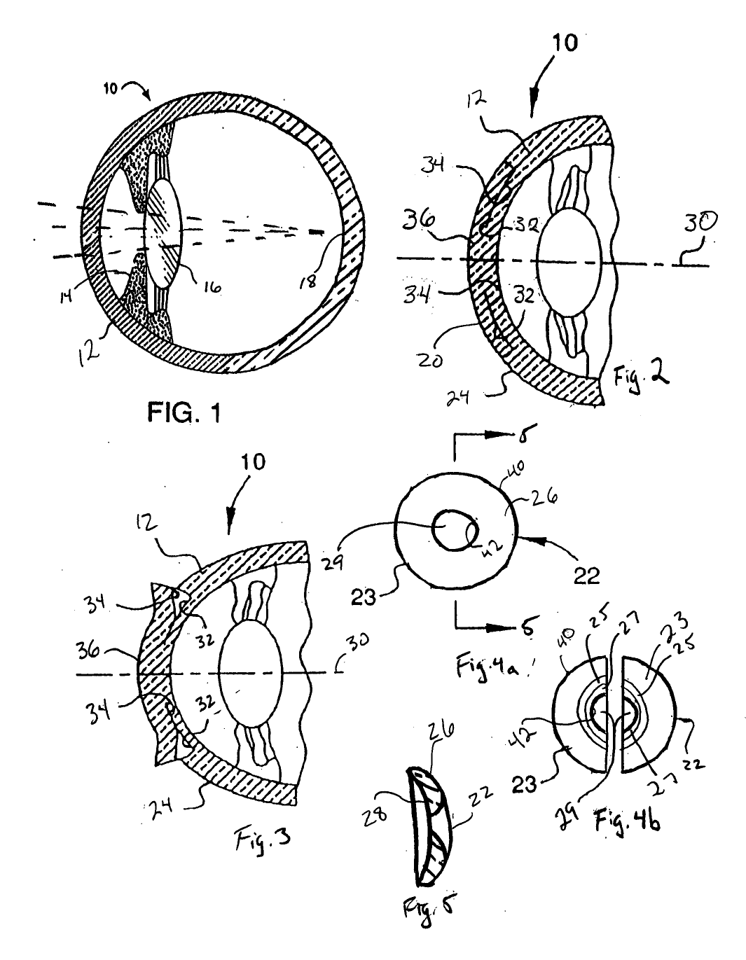

FIG. 1 is a side view in section taken through the center of an eye 10, which includes a cornea 12, a pupil 14 and a lens 16. As shown, the cornea 12 and lens 16 do not cooperatively focus light correctly on the retina of the eye to provide adequate vision. More specifically, light passing through the lens 16 of eye 10 is focused in front of the retina. To correct this refractive error, the curvature of the cornea can be modified to correct the refractive power of the cornea and thus correct the manner in which the light is focused with respect to the retina. It is noted that although a myopic eye is shown, each of the embodiments described herein is not limited to correction of this specific refractive error, and the present devices and methods can be used to correct any suitable error (e.g. myopia, hyperopia, presbyopia, astigmatism, any suitable combination thereof and any other focusing errors in the eye or combinations thereof).

As seen in FIGS. 1-6, the refractive properties of eye 10 can be altered by forming a flap 20 in the cornea 12 and then placing inlay, lens or implant 22 under flap 20. Implant 22 is positioned adjacent the exposed surface of the cornea and can be easily shaped or ablated using a laser, if desired. It is not necessary to ablate the implant 22, if the eye is properly corrected without ablation. Furthermore, the ablation can take place immediately upon placement on the surface of the cornea, or at any later time.

To begin, the refractive error in the eye is measured using wavefront technology, as is known to one of ordinary skill in the art. The refractive error measurements are used to determine the appropriate lens or implant 23 to best correct the error in the patient's cornea. Preferably, the lens 22 is manufactured or shaped prior to the use of the wavefront technology and is stored in a sterilized manner until that specific lens shape or size is needed. However, the information received during the measurements from the wavefront technology can be used to form the lens using a cryo-lathe, laser, or any other desired system, machine or device, or the lens can be shaped and stored in any suitable manner.

A flap or portion 20 can be formed in the surface 24 of the cornea 12, as seen in FIGS. 2 and 3. The flap can be formed using a laser, or it can be formed using a microkeratome as disclosed in U.S. Pat. No. 5,964,776 to Peyman, the entire contents of which are herein incorporated by reference. The flap may be formed be any means desired, such as with a knife, microkertome, or with a laser. Preferably an internal area of the cornea is separated into first 32 and second 34 substantially circular shaped internal surfaces to form the circular shaped corneal flap 20. First internal surface 32 faces in an anterior direction of cornea 12 and the second internal surface 34 faces in a posterior direction of the cornea 12. The flap 20 preferably remains attached to the cornea at a central portion or area 36 offset from the main optical axis 35. The flap 20 preferably has a uniform thickness of about 10-250 microns, and more preferably about 80-100 microns, but can be any suitable thickness. That is, the flap can be formed such that it separates layers of the stroma or layers of the epithelium, separates the epithelium from the Bowman's Layer, or separates the Bowman's layer from the stroma, or s formed in any other portion or suitable layer of the cornea. The flap can be formed in any suitable configuration, such a flap attached to the cornea at a location other than at the main optical axis or a flap that is not attached to the cornea at all. Additionally, the flap may be shaped or sized as desired and does not need to be circular or ring-shaped.

Intracorneal inlay, implant or lens 22 is preferably any polymer having about 50% water content; however, the water content can be any percentage desired. The lens may be formed from synthetic or organic material or a combination thereof. For example, the lens can be collagen combined with or without cells; a mixture of synthetic material and corneal stromal cells; silicone or silicone mixed with collagen; methylmetacrylate; any transparent material, such as polyprolidine, polyvinylpylidine, polyethylenoxyde, etc.; or any deformable polymer, which can change its shape with ablation after implantation, such as methacrylate and acrylic acid gel.

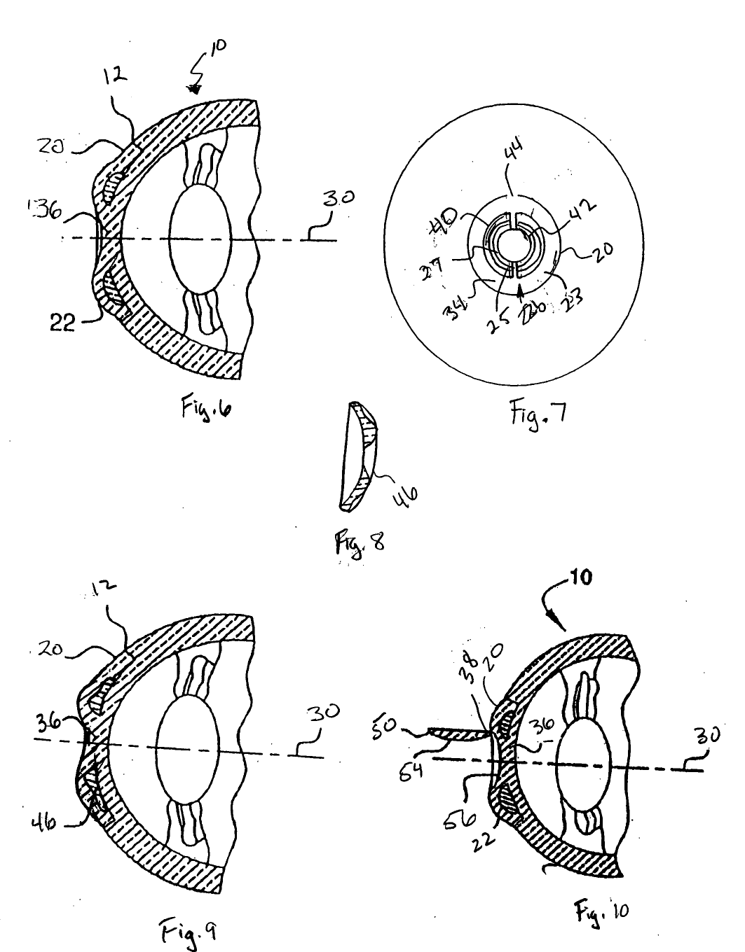

As shown in FIGS. 4a, 4b and 5, intracorneal implant 22 has a first surface 26 and a second surface 28. Implant 22 has a outer circumference or wall 40 and an inner circumference of wall 42. Wall 42 is substantially circular or arcuate and defines an opening 29. The inner radius is preferably between about 1.5 mm to about 2 mm and the outer radius is preferably between about 1.5 mm to about 10 mm. Additionally, implant 22 can be porous to allow oxygen and nutrients to pass therethrough. The thickness is preferably about 5-2000 microns, and more preferably less than 200 microns. The inside edge can be thinner or thicker than the outside edge; for example, the inside edge can have a thickness of about 1-100 microns, while the outside edge has a thickness of about 20-3000 microns. However, the intracorneal inlay 22 can have any thickness or configuration that would allow it to elevate or move any portion of surface 16 relative to surface 18. The thickness and position of intracorneal inlay 22 generally defines the degree of correction of the cornea.

Preferably, implant 22 is formed from an ablatable polymer and has at least one and more preferably several hundred physical openings or microperforations formed as passageways from the first surface of the inlay through the inlay to the second surface of the inlay. Each microperforation is about 0.1 microns to about 500 micros in diameter and extends from the first surface 26 to the second surface 28. These perforations form a net in the inlay, and permit molecules of oxygen, water, solute and protein to permeate through the inlay with substantially no or no inhibition. Any or all of the microperforations or openings in the any of the inlays described herein can have a glare-free material disposed thereon, if desired. For a further discussion of glare-free material, refer to U.S. Pat. Nos. 6,280,471 and 6,277,146 both to Peyman et al., the entire contents of both of which are incorporated herein by reference. It is noted that is not necessary to have either the perforations or the glare-free material describe herein.

As seen in FIG. 4a, implant 22 is preferably substantially ring-shaped; but can be a circular or semicircular inlay. For example, implant 22 can have a split (FIG. 4b) or have multiple portions that couple or fit together, it can be flat, arcuate, or tapered edges. Additionally, implant 22 may have any combination of these properties. Implant 22 can have multiple portions that can couple together, simply abut one another, they can lie near each other, not necessarily touching each other or the inlay portions can be separated from each other. Implant 22 can have multiple layers on top of each other, or have two sides with different thickness, which would help to correct astigmatism.

Additionally, the implant 22 preferably allows light in the visible spectrum to pass therethrough. The implant 22 can have refractive properties itself, and can have different or similar refractive properties to the refractive properties of the cornea. The inlay can have pigmentation added thereto to change the color of the implant 22 or it can be photochromatic. Furthermore, it is not necessary for the implant 22 to have a hole or aperture therethrough. The intracorneal inlay 22 can have a substantially planer surface or an arcuate surface with no holes or apertures therein. For additional configurations of inlays, see U.S. Pat. Nos. 6,063,073 and 6,217,571 both to Peyman, the entire contents of both of which are herein incorporated by reference.

Implant 22 can have substantially the same refractive index as the cornea or any other suitable index. For example, the implant 22 can have an index of refraction that is substantially higher than that of the cornea (i.e., up to about 1.76). Examples of suitable materials have been developed Nitto Denko Corporation and Brewer Science. Nitto Denko has increased the index of refraction of thermosetting resin by the addition of titania, zirconia and other metal oxide nanoparticles or the additional of titanium dioxide, zirconium dioxide and other metal oxide materials. Brewer Science has also developed a new approach to the preparation of hybrid coating systems where the high index metal oxide component forms spontaneously during the curing process of the coating, leaving the polymer and metal oxide phases at a near molecular-level of interdispersion. The resulting coatings have refractive indices ranging from 1.6 to as high as 1.9 (in the range of 400 to 700 nm) depending on the metal oxide loading. This high refractive index allows the lens to be thinner than a conventional lens, and still alter the refractive characteristics of the cornea. If formed from this material, the lens can have a thickness of between about 0.5 microns and 30 microns. Such a thickness allows the refractive properties of the eye to be altered using the refractive index of the lens and/or changing the curvature of the surface of the cornea.

As seen in FIG. 3, the flap 20 is then lifted using any device known in the art, such as spatula or microforceps or any other device, and implant 22 is positioned or introduced around or at least partially encircling the main optical axis 36 and between the first internal surface 32 and second internal surface 34 of the flap 20. However, as stated above, the flap does not necessarily have to be attached at the main optical axis, and in such a case implant 22 is merely placed under the flap. The flap 20 is then replaced so that it covers or lies over the implant 22 in a relaxed state, as seen in FIG. 6. In other words, implant 22 does not force flap 20 away from the internal surface 32 and therefore the refractive properties of the cornea are not altered due to a tension force being applied to the flap. Implant 22 is configured to change the corneal curvature at a predetermined area or portion of the cornea to alter the refractive properties thereof. In this specific example, the implant 22 is configured to flatten the corneal curvature and thereby correct myopia. However, implant 22 can be sized and configured to correct myopia, hyperopia, presbyopia, and astigmatism or any suitable combination thereof or any suitable combination of known vision disorders.

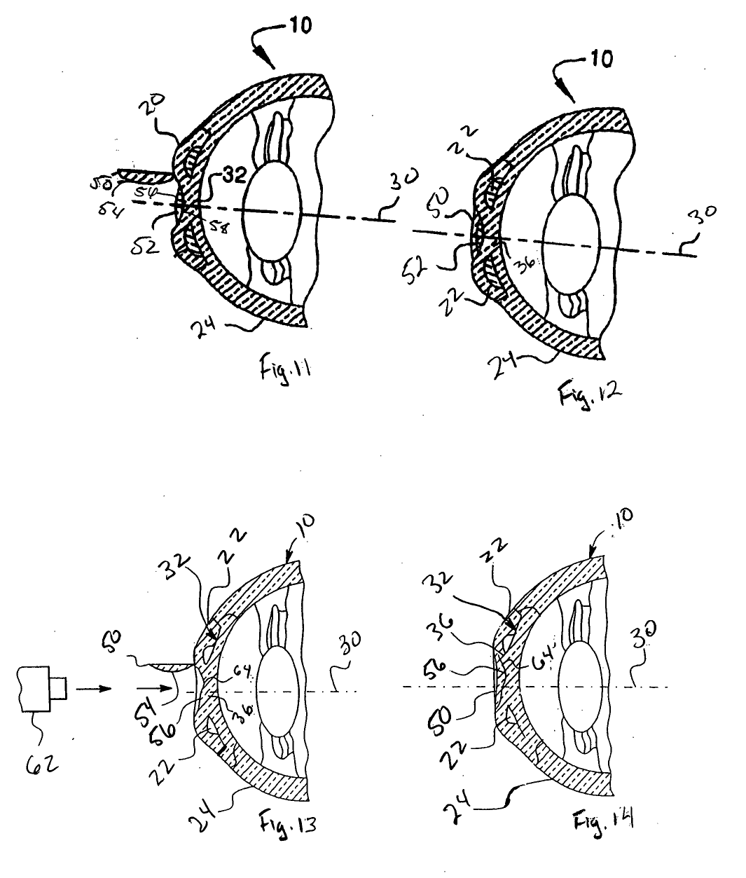

For example, the implant shown in FIGS. 4-6 is configured to correct myopia, presbyopia, and/or astigmatism. Preferably the implant corrects myopia by flattening out the corneal curvature or adding a minus diopter power. Furthermore, when the eye focuses on a near object the light rays passing through the central portion 36, and thus the opening 29 in the implant 22, the light rays are focused properly on the retina. This proper focusing is due to the fact that when the implant 22 changes the curvature of the cornea at an area offset from the main optical axis, there is residual change of the cornea at the central portion, thus correcting for presbyopia. For example, if the needed myopic correction is between about −2.0 diopters to about −5.0 diopters, an implant sized and configured to correct the myopia will also compensate for close vision or reading and thus have corrected the presbyopia. If the proper myopic correction is −2.0 diopters or less it is possible to insert an additional lens or ablate a portion of the cornea as discussed below for FIGS. 10-14.

If an eye is emmetropic and presbyopic an implant having several transition zones can be used, allowing the implant to have multifocal properties. As shown in FIG. 4b, the implant 22b preferably has three zones or portions: a first zone or portion 23, a second zone or portion 25 and a third zone or portion 27. The third portion is preferable adjacent the opening or arcuate portion 29 and has an index of refraction substantially similar to the cornea. Portion 27 is a transition zone and is configured to flatten the cornea at the central area, or the area adjacent the portion of the flat that remains attached to the cornea. The second portion 27 is a substantially ring-shaped portion that is radially further from the opening or center of the implant than portion 27. Portion 25 is also a transition zone and has an index of refraction higher that portion 27.

The first portion 23 is the optical zone and is steeper and has a higher index of refraction than both the second and third portions. In other words, the first zone is configured to alter the refractive properties of a first overlying area or portion of the cornea. This is preferably done by steepening the cornea to compensate for the refractive error in the eye, such as presbyopia. However, to maintain the emmetropic properties of the central portion of the cornea and thus allow the eye to view close objects, at least one of or both the second and third zones flatten the cornea (i.e., changing the curvature of the cornea) to compensate of the change in curvature by the first portion and/or change the refractive properties by having a different index of refraction. The implant does not necessarily need three zones or portions and can have as many or few as desired, for example, the implant can have one, two, three or more portions. Furthermore, the portions can be in any location desired and do not need to be concentric rings or arcs. It is noted that the above described refractive power changes can be effective on any type of implant and is not limited to implant 22b.

When inserting the implant between the layers, it is beneficial to have the interior radial portion or wall 42 sized and configured such that it can frictionally engage the area 36 attached to the cornea. Such an engagement will facilitate positioning and maintaining the position of the inlay relative to the corneal surface. This is particularly important when correcting astigmatic error.

Additionally, it is noted that an implant as shown in FIG. 4b can be used to facilitate correction of astigmatism. This is accomplished by aligning the axis of astigmatism along the line formed by positioning the two portions of the implant adjacent each other.

Furthermore, as shown in FIG. 7, a portion 44 of the periphery of the flap 20 can remain attached to the cornea 12. This also can facilitate correction of astigmatism, by forming portion 44 in such a position that portion 44. and the central area 36, are aligned along the astigmatic axis. Therefore, an implant such as implant 22b can be positioned such that the line formed by the two portions of implant 22b is aligned with the portion 44 and the area at the main optical axis 36. Since the implant 22b is configured to correct the astigmatic error when positioned in this manner, the positioning will be precise and there is less likelihood of error.

FIGS. 8 and 9 show an implant according to another embodiment of the present invention. Implant 46 is substantially similar to implant 22, except as seen specifically in FIG. 8, implant has a cross sectional shape that will steepen the comeal surface. This steepening effect will correct presbyopia with the implant, and will flatten the cornea at the central portion 36, thereby correcting myopia.

If necessary, or desired, the implant 22 can then be ablated by a laser beam that is activated outside the cornea and fired through the cornea to contact a portion of the inlay, or the flap 20 can be moved to the side and the inlay can be ablated directly. The ablating laser can be an excimer laser, which is generally known in the art for being capable of ablating both corneal tissue and synthetic material. However, since excimer lasers are generally developed for ablation of the cornea, there are expensive to produce, require toxic fluorine gases, and are difficult to maintain. Therefore, it may be preferable to ablate the implant 22 using lasers that are cheaper and easier to maintain. Certain lasers that produce a wavelength of about 355 nm can be cheaper and easier to maintain. However, it is noted that the laser can emit a beam having a wavelength of about 193 nm to about 1300 nm.

Preferably, when using this type of laser, the implant is ablated, producing holes in the polymer, without producing a coagulative effect on the material. The 355 nm photon has three times the energy of the conventional 1064 nm photon, enabling the 355 photon to break molecular bonds. The 355 nm wavelength can be generated using a diode pumped solid-state (DPSS) Nd-YAG laser, which is double frequencied to 532 nm and mixed with a Nd-YAG at 1064 nm, producing the 355 nm wavelength.

Additionally, the combination of a diffraction-linked beam and a short wavelength laser can enable machining of the implant, since the focal spot size is proportional to the wavelength. For example, the laser can emit a short pulse or ultrashort pulse of a picosecond, a nanosecond, a femtosecond or an attosecond. However, the laser can be any suitable continuous or pulsed laser, or any laser that emits a beam in the infrared or visual spectrum.

Preferably, when utilizing this type of frequency laser, a flying spot laser is used, which can be moved though a software program across the inlay to ablate the desired portion of the implant.

To further correct the refractive error in the cornea, a second flap 50 can be formed from the corneal epithelium on the surface 36 of the cornea 12 and a second inlay 52 can be placed under the second flap, a seen in FIGS. 10-12. The second inlay can be positioned under the second flap, during the same procedure (i.e., within minutes or seconds of the previous inlay) or at a later date or time (e.g., hours, days, weeks, months, or years later). Preferably, the flap is formed overlying portion 36 using a using alcohol, enzymes, such as condrotinase, plamin, alpha-chemotrypsin, pepsin, trypsin, or any other suitable enzyme, a laser, such as an attosecond or femtosecond laser, a microkeratome or a knife.

When alcohol is used, the alcohol loosens the epithelium from the basement membrane, which allows removal of the epithelial layer. Heating the alcohol solution can also loosen the epithelium and facilitate removal. It is noted that any of the herein described solutions, such as the enzyme solutions can also be heated to facilitate removal of the epithelium. Preferably, the alcohol is heated to between about 40° C. and about 50° C., and more preferably to about 47° C. The flap can also be formed to remain at least partially attached to the cornea, as shown in FIGS. 10 and 11, by a portion 38 that allows the second flap to be positioned in the proper orientation, if it is desired to have flap repositioned over the second inlay 52. The flap has a first surface 54 and a second surface 46. The first surface 54 faces in a posterior direction of the eye the second surface 56 faces in an anterior direction of the eye

The second flap 50 is a relatively small flap that preferably at least partially overlies or is concentric about the visual axis or main optical axis 30 and can be attached to the cornea 12 by portion 38. However, the flap can be formed on any portion of the cornea desired and in any suitable manner, such as with alcohol, a knife, blade or laser, as discussed above. It is noted that the location of the flap does not necessarily need to be concentric about the main optical axis and can be at any location on the surface of the eye and may be any size desired.

The flap is preferably pealed or moved away from the surface of the cornea using a suction device, microfoceps, or using any other device known in the art. For a further discussion of the formation of this type of flap, see U.S. patent application Ser. No. 09/843,141, filed Apr. 27, 2001, the entire contents of which are incorporated herein by reference.

Once the flap is moved to expose surfaces 54 and 56, second intracorneal inlay, implant or lens 52 can be positioned adjacent one of the surfaces. As shown in FIGS. 11 and 12, implant 52 is a generally convex lens (for correction of hyperopia), with a first surface 56 and a second surface 58, and has a diameter that is smaller than the diameter of implant 22; however, implant 52 can be any suitable size or configuration desired. For example, implant 52 can have a concave, convex-concave or plano-convex or toric surface, or any other configuration described above.

Implant 52 preferably is formed or a pliable material that conforms to the surface of the eye, and is ablatable by a laser, as described below; however, implant 52 can be formed from any of the materials described above for implant 22, or any other suitable material. For example, implant 52 can be formed from any ablatable polymer, methacrolate and methocrolate gel, acrylic acid, polyvinylprolidine, silicone or a combination of the these materials or a combination of these materials with an organic material, such as collegen, chondrotine sulfate, glycosamine glycon, integrin, vitronectin, fibronnectine and/or mucopoly saccaride. Each of these materials and/or any combination thereof can also be used for implant 22, described above. It should be noted that implant 52 does not necessarily need to be positioned in the cornea after implant 22 and can be positioned in the cornea prior to implant 22.

Furthermore, implant 52 can be a substantially ring-shaped inlay (for the correction of myopia) and can be formed from any of the materials, have any of the configurations and/or dimensions of implant 22.

Implant 52 can have openings or microperforations therein, which permit molecules of oxygen, water, solute and protein to permeate through the inlay with substantially no or no inhibition. Such microperforations are substantially similar to the microperforations described above and any description thereof is applicable to these microperforations.

As seen in FIGS. 11 and 12, implant 52 is preferably positioned closer to the surface of the cornea than implant 22. Additionally, since implant 52 has a diameter that is smaller than implant 22, and implant 22 preferably has an opening therein, an axis or line drawn substantially parallel the main optical axis through the second implant can pass through the opening of the first implant, and not pass through the implant itself. However, as noted above, implant 52 can have an opening therein, and in such a procedure the main optical axis of the eye can pass through the opening in each implant.

Since each implant has micro perforations, an excimer laser can be readily used to ablate the implants, and will not cause irregularities in the surface. Each implant can be filled with water or glycosamine glycon from the cornea, which will leave similar ablation characteristics as the cornea. In addition, the spot size used for ablation will generally be larger than the diameter of each perforation, and therefore at least a portion of the implant will be ablated. Furthermore, since the corneal epithelium cells are generally larger than the microperforations, the cornea epithelium will straddle the microperforation.

After the procedure, a short-term bandage contact lens may also be used to protect the cornea, and keep the second implant stable. Preferably, the contact covers the implant inlay; however, the contact may be large enough to cover the area defined by each implant and/or either or both flaps.

Additionally, if desired, second implant 52 can be ablated with an excimer laser or any other laser described above for the ablation of the first implant 22. The flap is then positioned over the implant (either ablated or unablated) without tension as described for flap 20, as seen in FIG. 12.

By performing the above described procedure using two separate components or inlays, the size or thickness of each inlay can be reduced, which reduces the inhibition of the flow of nutrients through the system in general.

Additionally, the refractive properties of the system can be adjusted after the procedure has been completed. For example, either or both of the inlays can be ablated using a laser after implantation. If desired, the flaps can be reopened and moved to expose the desired inlay, so that the inlay can be ablated directly, or the laser can ablate the inlay through the cornea epithelium. Furthermore, the refractive properties can be altered by replacement of one or both of the inlays. Since the adhesion between the inlays and cornea are not strong in the present procedures, one or both of the inlays can be readily replaced at anytime without the risk of a potential scar on the cornea.

In a further embodiment, second flap 50 can be formed from the corneal epithelium on the surface 24 of the cornea 20 (or in any other suitable portion or layer of the cornea), a seen in FIGS. 13 and 14 to reduce or eliminate irregularities in the healing of the cornea.

The flap is preferably pealed or moved away from the surface of the cornea using a suction device (not shown), but may be removed using any other device known in the art.

Once the flap is moved to expose surfaces 54 and 56, an excimer laser 62, as seen in FIG. 12, can be used to ablate a portion 64 of the cornea 20 to reduce or eliminate any remaining refractive error. Portion 64 is preferably a portion of the Bowman's layer or basement membrane, but can be any portion of the cornea desired. The flap 50 is then replaced and allowed to heal as seen in FIG. 13. The flap may simply be placed over the ablated portion and heal or it may be affixed thereto in any manner known in the art, such as by sutures or adhesive.

When performing the excimer laser procedures described above, it is possible to simultaneously use wavefront technology or Adaptec optic technology to create a near perfect correction in the eye and to remove all corneal irregularities. By using this technique to correct vision, it is possible to achieve 20/10

vision in the patient's eye or better.

The patient can undergo the second laser ablation, as seen in FIGS. 12, either immediately after the insertion of the ocular implant or after a substantial time difference, such as days or weeks later, and any step or portion of the above procedure may be repeated to decrease the refractive error in the eye.

Furthermore, at the end of the procedure or before the ablation of the surface of the cornea, topical agents, such as an anti-inflammatory, antibiotics and/or an antiprolifrative agent, such as mitomycin or thiotepa, at very low concentrations can be used over the ablated area to prevent subsequent haze formation. The mitomycin concentration is preferably about 0.005-0.05% and more preferably about 0.02%. A short-term bandage contact lens may also be used to protect the cornea. The short term contact lens specifically protects the portion of the cornea that has flap 50 formed thereon, but also can protect the cornea after any of the above steps in this procedure.

While preferred embodiments have been chosen to illustrate the invention, it will be understood by those skilled in the art that various changes and modifications can be made therein without departing from the scope of the invention as defined in the appended claims.

1. A method of changing the refractive properties of an eye, comprising the steps of
separating the cornea to form a first layer and a second layer, said first layer facing in a posterior direction of the eye and said second layer facing in an anterior direction of the eye, said first and second layers remaining attached at an area at the main optical axis,
inserting an implant between said first and second layers, said implant including a first portion having a first index of refraction and adapted to change the refractive properties of a first portion of the eye, a second portion having a second index of refraction and adapted to change the refractive properties of a second portion of the eye, and a third portion having a substantially arcuate edge adapted to be positioned adjacent the area attached at the main optical axis.

2. A method according to claim 1 wherein
said implant is substantially ring-shaped and said first portion forms a substantially ring-shaped portion located at the periphery of said implant.

3. A method according to claim 1, wherein
said implant is substantially ring-shaped and shaped third portion substantially surrounds said area attached at the main optical axis.

4. A method according to claim 1, wherein
said first index of refraction is higher than said second index of refraction.

5. A method according to claim 1, wherein
said third portion has a third index of refraction, said third index of refraction being lower than said second index of refraction.

6. A method according to claim 5, wherein
said third index of refraction is substantially the same as the corneal index of refraction.

7. A method according to claim 6, wherein
said third portion is a transition zone, which flattens the cornea adjacent the area attached at the main optical axis.

8. A method according to claim 1, wherein
said step of separating the cornea includes forming a flap in the corneal stroma.

9. A method according to claim 1, wherein
said step of separating the cornea includes forming an epithelial flap.

10. A method according to claim 1, further comprising the step of
ablating a surface of the cornea overlying the area attached at the main optical axis.

11. A method according to claim 10, wherein
said surface overlying the main optical axis is a surface exposed by at least one of a corneal flap and a stromal flap.

12. An implant for changing the refractive properties of an eye, comprising:
a body adapted to be implanted between layers of the cornea offset from the main optical axis, thereby changing the refractive properties of the eye, the body including
a first portion having a first refractive power and configured to change the refractive properties of a first area of the cornea by changing the corneal curvature thereof; and
a second portion having a second refractive power, said second refractive power configured to change the refractive properties of a second area of the cornea and 4 compensate for error at said second area caused by said first portion.

13. An implant according to claim 12, wherein
said first portion is configured to have a first refractive index; and
said second portion is configured to have a second refractive index, said second refractive index being lower than said first index.

14. An implant according to claim 12, wherein
said body is substantially ring-shaped and said first portion is a substantially ring-shaped portion located at the periphery of said body portion.

15. An implant according to claim 13, wherein
said body further includes a third portion having a third refractive index, said third refractive index being less than said second refractive index and being substantially the same as the refractive index of the cornea.

16. An implant according to claim 15, wherein
said third portion is a transition zone configured to flatten a predetermined portion of the cornea.

17. An implant for changing the refractive properties of an eye, comprising:
a first semi circular body portion adapted to be implanted between layers of the cornea offset from the main optical axis, thereby changing the refractive properties of the eye, the first semi circular body portion including
a first portion having a first refractive power and configured to change the refractive properties of a first area of the cornea by changing the corneal curvature thereof; and
a second portion having a second refractive power, said second refractive power configured to change the refractive properties of a second area of the cornea and compensate for error at said second area caused by said first portion; and
a second semi circular body portion adapted to be implanted between layers of the cornea offset from the main optical axis, thereby changing the refractive properties of the eye, the second semi circular body portion including
a third portion having a third refractive power configured to change the refractive properties of a third area of the cornea by changing the corneal curvature thereof; and
a fourth portion having a fourth refractive power, said fourth refractive power configured to change the refractive properties of a fourth area of the cornea and compensate for error at said second area caused by said third portion.

18. A method of changing the refractive properties of the an eye, comprising the steps of
separating the cornea to form a first layer and a second layer, said first layer facing in a posterior direction of the eye and said second layer facing in an anterior direction of the eye, said first and second layers remaining attached at an area at the main optical axis,
inserting a first semi-circular implant between said first and second layers, said first implant including a first portion having a first index of refraction and adapted to change the refractive properties of a first portion of the eye, and a second portion having a second index of refraction and adapted to change the refractive properties of a second portion of the eye; and
inserting a second semi-circular implant between said first and second layers, said second implant including a third portion having a third index of refraction and adapted to change the refractive properties of a third portion of the eye, a fourth portion having a fourth index of refraction and adapted to change the refractive properties of a fourth portion of the eye.

19. A method according to claim 18, further comprising the step of
positioning the first and second implants such that they are aligned with an astigmatic axis of the eye.

20. A method according to claim 19, wherein
the step of separating the cornea to form a first corneal layer and a second corneal layer includes separating the cornea to form a substantially circular flap that is coupled to the cornea at substantially the center thereof and disconnected at substantially all of the periphery thereof.

21. A method according to claim 20, wherein
the step of separating the cornea to form a first corneal layer and a second corneal layer includes allowing a portion of the flap to remain attached at the periphery thereof, said portion of the flap remaining attached to the periphery and the area attached at the main optical axis are configured to be aligned with the astigmatic axis of the eye.
